(19) United States
(12) Patent Application Publication
(10) Pub. No.: US 2022/0257133 A1
(43) Pub. Date: Aug. 18, 2022

(54) PULSE DIAGNOSIS DEVICE AND CONTROL METHOD THEREFOR

(71) Applicant: Beijing Taiya Technology Co., Ltd., Beijing (CN)

(72) Inventors: Be XIE, Beijing (CN); Changhong YU, Beijing (CN)

(21) Appl. No.: 17/600,841

(22) PCT Filed: Apr. 2, 2020
(86) PCT No.: PCT/CN2020/082954

§ 371 Date: May 2, 2022

(30) Foreign Application Priority Data
Apr. 4, 2019 (CN) 201910272287.6
Apr. 4, 2019 (CN) 201920454474.1

(51) Int. Cl. A61B 5/024

(52) U.S. Cl. CPC A61B 5/02438; A61B 5/02444

(57) ABSTRACT

A pulse diagnosis device and a control method therefor, relating to the technical field of pulse diagnosis, and specifically aiming to solve the problem that an existing pulse diagnosis device cannot be well adapted to wrists of different sizes. The pulse diagnosis device comprises a housing (1), a cavity (2) which is formed in the housing (1) and accommodates a wrist (8), an airbag assembly provided between the cavity (2) and an inner wall of the housing (1), a controller (6) and an air pump assembly connected to the airbag assembly; the airbag assembly comprises one or more first airbags (31) and a second airbag (32) which are sequentially stacked from outside to inside, the first airbags (31) and the second airbag (32) each are provided with an air pressure sensor (53), a pulse diagnosis sensor (4) is provided on the side of the second airbag (32) facing the cavity (2), and the controller (6) can control the air pump assembly to inflate any one of the first airbags (31) and the second airbag (32) separately. The control method comprises first inflating the first airbags (31), and then inflating the second airbag (32), so that the airbag assembly has a wide range of deformation amounts to adapt to wrists (8) of different sizes, improving the comfort and pulse diagnosis accuracy.

### FIELD

The present disclosure relates to the technical field of pulse diagnosis, and specifically provides a pulse diagnosis instrument and a control method therefor.

### BACKGROUND

With the continuous development and progress of science and technology, more and more medical devices have been developed. The development and application of a pulse diagnosis instrument have greatly improved the level of diagnosis and treatment of Chinese medicine. The pulse diagnosis instrument is typically provided with an airbag in its shell, and a sensor is arranged on the airbag. The airbag is inflated/deflated so as to be deformed so that the sensor comes into or out of contact with a wrist. Then, the sensor collects pulse condition information when it contacts the wrist. By collecting the pulse condition information by the sensor, the accuracy of obtaining the pulse condition information is improved, thus preventing doctors from making incorrect judgments on the health condition of human body due to inaccurate pulse condition information.

However, for people whose wrists are too thin, the airbag of the pulse diagnosis instrument cannot make the sensor abut against the wrist in a completely adhering state after the airbag is inflated, and for people whose wrists are too thick, a great pressure is applied to the wrist by the sensor after the airbag of the pulse diagnosis instrument is inflated, which causes discomfort of the wrist. That is to say, the existing pulse diagnosis instruments have a problem that they cannot well adapt to wrists of different thicknesses.

Accordingly, there is a need in the art for a new technical solution to solve the above problem.

### SUMMARY

In order to solve the above problem in the prior art, that is, to solve the problem that the existing pulse diagnosis instruments cannot well adapt to wrists of different thicknesses, in an aspect, the present disclosure provides a pulse diagnosis instrument, which includes a shell, a chamber formed in the shell to accommodate a wrist, an airbag assembly provided between the chamber and an inner wall of the shell, a controller, and an air pump assembly connected to the airbag assembly, in which the airbag assembly includes one or more first airbags and a second airbag stacked in sequence from the outside to the inside, the first airbags and the second airbag are each equipped with an air pressure sensor, the second airbag is provided with a pulse diagnosis sensor on a side facing the chamber, and the controller can control the air pump assembly to separately inflate any one of the first airbags and the second airbag.

In a preferred technical solution of the above pulse diagnosis instrument, the air pump assembly includes a plurality of first air pumps, each of the plurality of first air pumps is connected to each of the first airbags and the second airbag in a one-to-one correspondence, and the first air pumps are configured to at least inflate the first airbags and the second airbag.

In a preferred technical solution of the above pulse diagnosis instrument, the air pump assembly further includes a plurality of second air pumps, each of the plurality of second air pumps is also connected to each of the first airbags and the second airbag in a one-to-one correspondence, and the second air pumps are configured to accelerate the deflation of the first airbags and the second airbag.

In a preferred technical solution of the above pulse diagnosis instrument, the first airbags and the second airbag are each provided with an air inlet-and-outlet port, and each of the air inlet-and-outlet ports is connected to a corresponding first air pump and second air pump respectively through a three-way valve; or the first airbags and the second airbag are each provided with an air inlet and an air outlet, the air inlet is connected to a corresponding first air pump, and the air outlet is connected to a corresponding second air pump.

In a preferred technical solution of the above pulse diagnosis instrument, the first air pumps are dual-purpose pumps capable of both inflating and deflating.

In a preferred technical solution of the above pulse diagnosis instrument, each of the first airbags is arranged around the chamber.

In a preferred technical solution of the above pulse diagnosis instrument, each of the first airbags includes a plurality of communicating inflatable cavities.

In a preferred technical solution of the above pulse diagnosis instrument, the airbag assembly is equipped with a restoring unit to accelerate the speed of the first airbag restoring to an initial state during the deflation process.

In a preferred technical solution of the above pulse diagnosis instrument, the restoring unit includes an elastic member arranged between the innermost first airbag and the second airbag; or the restoring unit includes a plurality of elastic members arranged between the innermost first airbag and the second airbag as well as between the plurality of first airbags.

In a preferred technical solution of the above pulse diagnosis instrument, the elastic member is an arc-shaped elastic strip with both ends overlapped.

It can be understood by those skilled in the art that in the technical solutions of the present disclosure, by providing one or more first airbags and a second airbag stacked in sequence from the outside to the inside between the chamber for accommodating the wrist and the inner wall of the shell, the controller can control the air pump assembly to separately inflate any one of the first airbags and the second airbag. Through such an arrangement, each of the airbags, without much gas filling, enables the airbag assembly to have a larger deformation, so there is a larger deformation range. Moreover, a curvature of the surface of the second airbag is small after inflation, so the second airbag can well fit with the wrist, and the forces on the wrist are relatively uniform, thus avoiding the problems with the pulse diagnosis instrument with only one airbag that it cannot be adapted to wrists with different thicknesses when the deformation range of the airbag is small, and that when the deformation range of the airbag is large, the airbag has a large curvature of the surface after inflation, which causes uneven forces on the wrist, thus affecting the accuracy of the pulse diagnosis sensor and the comfort of the wrist. Therefore, the present disclosure can be adapted to wrists with different thicknesses, expand the application range of the pulse diagnosis instrument, and solve the problem that the existing pulse diagnosis instruments cannot be well adapted to wrists of different thicknesses. In addition, the cooperation of the first airbags and the second airbag enables the pulse diagnosis sensor to abut against a radial artery measurement area of the wrist with an appropriate force, which improves the clamping comfort of the airbag assembly to the wrist on the basis of ensuring the accuracy of collecting pulse condition information.

In the preferred technical solutions of the present disclosure, the air pump assembly includes a plurality of first air pumps, each of the first air pumps is connected to each of the first airbags and the second airbag in a one-to-one correspondence, and the first air pumps are configured to at least inflate the first airbags and the second airbag. By connecting a plurality of first air pumps to the first airbags and the second airbag in a one-to-one correspondence, the first airbags and the second airbag can be inflated at the same time, the inflation duration is shortened, the inflation efficiency is improved, and the user experience is optimized.

Preferably, the air pump assembly further includes a plurality of second air pumps, each of the second air pumps is also connected to each of the first airbags and the second airbag in a one-to-one correspondence, and the second air pumps are configured to accelerate the deflation of each of the first airbags and the second airbag. Through the arrangement of a plurality of second air pumps, the deflation speeds of the first airbags and the second airbag can be accelerated, and the first airbags and the second airbag can be acceleratedly restored to the initial state after the pulse diagnosis sensor acquires the pulse condition, so that the wrist can move out of the chamber in time, which further optimizes the user experience.

In another aspect, the present disclosure also provides a control method for a pulse diagnosis instrument, in which the pulse diagnosis instrument includes a shell, a chamber formed in the shell to accommodate a wrist, an airbag assembly provided between the chamber and an inner wall of the shell, a controller, and an air pump assembly connected to the airbag assembly; the airbag assembly includes one or more first airbags and a second airbag stacked in sequence from the outside to the inside, the first airbags and the second airbag are each equipped with an air pressure sensor, and the second airbag is provided with a pulse diagnosis sensor on a side facing the chamber; and the control method includes the following steps: controlling, by the controller, the air pump assembly to inflate the first airbags to a clamping pressure; controlling, by the controller, the air pump assembly to inflate the second airbag to a pulse diagnosis pressure; controlling, by the controller, the pulse diagnosis sensor to collect pulse condition information of the wrist; and controlling, by the controller, the first airbags and the second airbag to deflate.

In a preferred technical solution of the above control method, the step of “controlling by the controller the air pump assembly to inflate the first airbags to the clamping pressure” specifically includes: controlling, by the controller, the air pump assembly to sequentially inflate each of the first airbags to the clamping pressure in an order from the outside to the inside.

In a preferred technical solution of the above control method, the step of “controlling by the controller the first airbags and the second airbag to deflate” specifically includes: controlling, by the controller, the first airbags and the second airbag to deflate at the same time.

In a preferred technical solution of the above control method, the step of “controlling by the controller the first airbags and the second airbag to deflate” specifically includes: controlling, by the controller, the second airbag and the first airbags to sequentially deflate in an order from the inside to the outside.

In a preferred technical solution of the above control method, the air pressures of the first airbags and the air pressure of the second airbag are the same after deflation.

In addition, the present disclosure also provides a control method for a pulse diagnosis instrument, in which the pulse diagnosis instrument includes a shell, a chamber formed in the shell to accommodate a wrist, an airbag assembly provided between the chamber and an inner wall of the shell, a controller, and an air pump assembly connected to the airbag assembly; the airbag assembly includes one or more first airbags and a second airbag stacked in sequence from the outside to the inside, the first airbags and the second airbag are each equipped with an air pressure sensor, and the second airbag is provided with a pulse diagnosis sensor on a side facing the chamber; and the control method includes the following steps: controlling, by the controller, the air pump assembly to inflate the first airbags to a first set pressure; controlling, by the controller, the air pump assembly to inflate the second airbag to a second set pressure; controlling, by the controller, the air pump assembly to inflate the first airbags so that an air pressure of the second airbag reaches a pulse diagnosis pressure; controlling, by the controller, the pulse diagnosis sensor to collect pulse condition information of the wrist; and controlling, by the controller, the first airbags and the second airbag to deflate.

It should be noted that the control method for the pulse diagnosis instrument has all the technical effects of the pulse diagnosis instrument described above, which will not be described repeatedly.

### BRIEF DESCRIPTION OF DRAWINGS

Preferred embodiments of the present disclosure will be described below with reference to the accompanying drawings, in which:

FIG. 1 is a first schematic structural view of a pulse diagnosis instrument according to an embodiment of the present disclosure (in which airbags are in an initial state);

FIG. 2 is a second schematic structural view of a pulse diagnosis instrument according to an embodiment of the present disclosure (in which the airbags are in a working state);

FIG. 3 is a schematic view showing connection relationships between an airbag assembly, an air pump, a solenoid valve, an air pressure sensor, a three-way valve, and a controller in a pulse diagnosis instrument according to an embodiment of the present disclosure;

FIG. 4 is a schematic structural view of first airbags in a pulse diagnosis instrument according to an embodiment of the present disclosure;

FIG. 5 is another schematic structural view of first airbags in a pulse diagnosis instrument according to an embodiment of the present disclosure;

FIG. 6 is a schematic view showing steps of a control method for a pulse diagnosis instrument according to an embodiment of the present disclosure; and

FIG. 7 is a schematic view showing a change of pressure over time in a second airbag under the control of another control method for the pulse diagnosis instrument of the present disclosure.

### LIST OF REFERENCE SIGNS

1: shell; 2: chamber; 31: first airbag; 311: inflatable cavity; 32: second airbag; 4: pulse diagnosis sensor; 51: inflating air pump; 52: miniature vacuum pump; 53: air pressure sensor; 54: three-way valve; 55: solenoid valve; 6: controller; 7: arc-shaped elastic strip; 8: wrist; 81: radial artery blood vessel.

### DETAILED DESCRIPTION

Preferred embodiments of the present disclosure will be described below with reference to the accompanying drawings. It should be understood by those skilled in the art that these embodiments are only used to explain the technical principles of the present disclosure, and are not intended to limit the scope of protection of the present disclosure. For example, although the number of the first airbags in the pulse diagnosis instrument of the present disclosure is 3, those skilled in the art can make adjustment thereto as required so as to adapt to specific applications. For example, the number of the first airbags in the pulse diagnosis instrument of the present disclosure may be 1, 2, 4, 5 or more. Obviously, the adjusted technical solutions will still fall within the scope of protection of the present disclosure.

It should be noted that in the description of the present disclosure, terms indicating directional or positional relationships, such as “left”, “right”, “upper”, “lower”, “inner”, “outer” and the like, are based on the directional or positional relationships shown in the accompanying drawings. They are only used for ease of description, and do not indicate or imply that the device or element must have a specific orientation, or be constructed or operated in a specific orientation, and therefore they should not be considered as limitations to the present disclosure. In addition, terms “first” and “second” are merely used for description, and should not be construed as indicating or implying relative importance.

In addition, it should also be noted that in the description of the present disclosure, unless otherwise clearly specified and defined, terms “arrange” and “connect” should be understood in a broad sense; for example, the connection may be a fixed connection, or may also be a detachable connection, or an integral connection; it may be a mechanical connection, or an electrical connection; it may be a direct connection, or an indirect connection implemented through an intermediate medium, or it may be an internal communication between two elements. For those skilled in the art, the specific meaning of the above terms in the present disclosure can be understood according to specific situations.

In addition, in order to better illustrate the present disclosure, numerous specific details are given in the following specific embodiments. It should be understood by those skilled in the art that the present disclosure may also be implemented without certain specific details. In some embodiments, methods, means, elements, and circuits that are well known to those skilled in the art are not described in detail in order to highlight the spirit of the present disclosure.

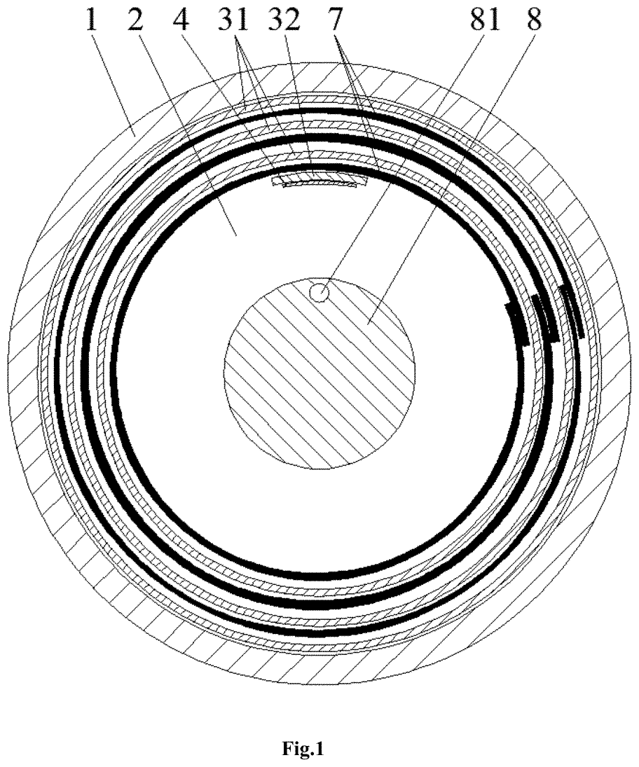

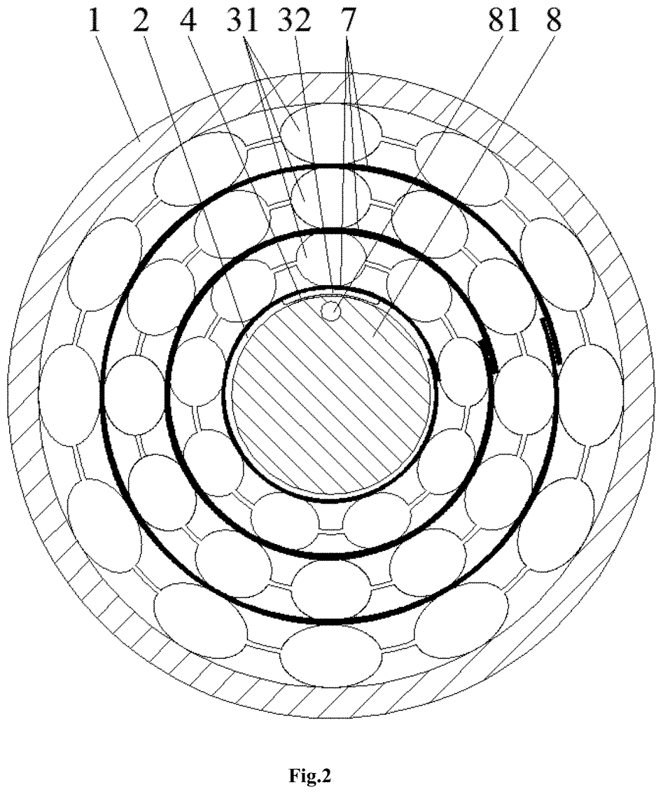

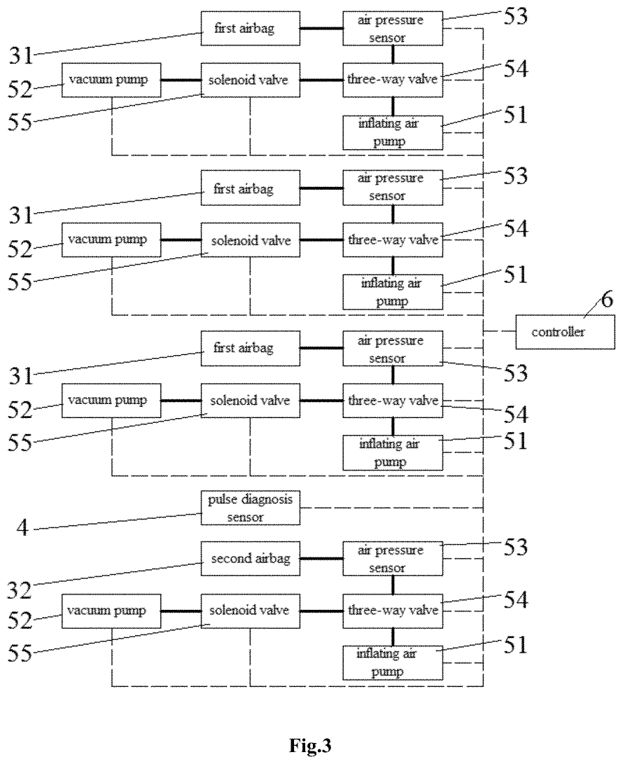

Reference is made to FIGS. 1 to 3, FIG. 1 is a first schematic structural view of a pulse diagnosis instrument according to an embodiment of the present disclosure (in which airbags are in an initial state); FIG. 2 is a second schematic structural view of a pulse diagnosis instrument according to an embodiment of the present disclosure (in which the airbags are in a working state); and FIG. 3 is a schematic view showing connection relationships between an airbag assembly, an air pump, a solenoid valve, an air pressure sensor, a three-way valve, and a controller in a pulse diagnosis instrument according to an embodiment of the present disclosure.

As shown in FIGS. 1 to 3, the pulse diagnosis instrument includes a shell 1. A chamber 2 for accommodating a wrist 8 is formed in the shell 1, and an airbag assembly is provided between the chamber 2 and an inner wall of the shell 1. The pulse diagnosis instrument also includes a controller 6, and an air pump assembly connected to the airbag assembly. The airbag assembly includes three first airbags 31 and one second airbag 32 stacked in sequence from the outside to the inside. The first airbags 31 and the second airbag 32 are respectively equipped with an air pressure sensor. For example, air pressure sensors 53 are provided in pipelines communicating the air pump assembly with the first airbags 31 and the second airbag 32 respectively to detect air pressures in the first airbags 31 and the second airbag 32 in real time. A pulse diagnosis sensor 4 is provided on a side of the second airbag 32 that faces the chamber 2 (i.e., a lower side of the second airbag 32 in the orientation shown in FIG. 1). The controller 6 can control the air pump assembly to separately inflate any one of the three first airbags 31 and the one second airbag 32. It should be noted that the chamber 2 is a space that changes as a volume of the airbag assembly changes. The size of the shell 1 may be set according to the actual situation. For example, an inner diameter of the shell 1 may be set to 50 mm to 100 mm, an outer diameter may be set to 50 mm to 150 mm, with the outer diameter being larger than the inner diameter, and a height of the shell 1 (i.e., the size of the shell 1 in the axial direction) may be set to 50 mm to 150 mm.

Specifically, the air pump assembly includes four first air pumps, such as inflating air pumps 51, and each of the first airbags 31 and the second airbag 32 is connected to one of the inflating air pumps 51 and a solenoid valve 55 through a three-way valve 54 respectively. The controller 6 is communicatively connected to the inflating air pumps 51, the air pressure sensors 53, the pulse diagnosis sensor 4 and the solenoid valves 55. After the patient's wrist 8 extends into the chamber 2, the controller 6 controls the inflating air pumps 51 connected to the three first airbags 31 to work in a specific sequence, so as to inflate the three first airbags 31 respectively. For example, the controller 6 controls the inflating air pump 51 connected to the outermost first airbag 31 to work and inflate. During the inflation process, the corresponding air pressure sensor 53 detects the air pressure value of the outermost first airbag 31 in real time. When the air pressure value reaches a set air pressure value, inflation of the outermost first airbag 31 is stopped. Then, the controller 6 controls the inflating air pump 51 connected to the middle first airbag 31 to work and inflate. During the inflation process, the corresponding air pressure sensor 53 detects the air pressure value of the middle first airbag 31 in real time. When the air pressure value reaches a set air pressure value, inflation of the middle first airbag 31 is stopped. Then, the controller 6 controls the inflating air pump 51 connected to the innermost first airbag 31 to work and inflate. During the inflation process, the corresponding air pressure sensor 53 detects the air pressure value of the innermost first airbag 31 in real time. When the air pressure value reaches a set air pressure value, inflation of the innermost first airbag 31 is stopped so that the innermost first airbag 31 abuts against a surface of the wrist 8 at a suitable pressure. Finally, the controller 6 controls the inflating air pump 51 connected to the second airbag 32 to inflate so that the air pressure in the second airbag 32 reaches a set value. The second airbag 32 presses the pulse diagnosis sensor 4 on an area corresponding to a radial artery vessel 81 on the wrist 8 at an appropriate pressure. After the pulse diagnosis sensor 4 collects the pulse condition information, the controller 6 controls the solenoid valves 55 to open, thereby deflating the first airbags 31 and the second airbag 32 to restore the airbag assembly to the initial state (that is, the state before inflation), so that the patient's wrist 8 moves out of the chamber 2 and is ready for the next pulse diagnosis operation.

The airbag assembly provided between the inner wall of the shell 1 and the chamber 2 includes one or more first airbags 31 and one second airbag 32 stacked in sequence from the outside to the inside, and each of the airbags enables the airbag assembly to have a larger deformation without being inflated with much gas, so there is a larger deformation range. Moreover, the curvature of the surface of the second airbag is small after inflation, so the second airbag can well fit with the wrist, and the forces on the wrist are relatively uniform, thus avoiding the problems with the pulse diagnosis instrument with only one airbag that it cannot be adapted to wrists with different thicknesses when the deformation range of the airbag is small, and that when the deformation range of the airbag is large, the airbag has a large curvature of the surface after inflation, which causes uneven forces on the wrist, thus affecting the accuracy of the pulse diagnosis sensor and the comfort of the wrist. Therefore, the present disclosure can be better adapted to wrists with different thicknesses, expand the application range of the pulse diagnosis instrument, and solve the problem that the existing pulse diagnosis instruments cannot be well adapted to wrists of different thicknesses. In addition, the controller 6 can control the air pump assembly to inflate the first airbags 31 and the second airbag 32 in stages, so that the first airbags 31 abut against the surface of the wrist 8 at suitable pressures, thereby fixing the wrist 8 in a more comfortable way and optimizing the use experience; moreover, the second airbag 32 is pressed against the area corresponding to the radial artery vessel 81 on the wrist 8 at a suitable pressure, which greatly improves the accuracy of collecting the pulse condition information by the pulse diagnosis sensor 4.

It can be understood by those skilled in the art that the number of the first airbags 31 in the airbag assembly being 3 is only an exemplary description, and those skilled in the art can make adjustment as required so as to adapt to specific applications. For example, the number of the first airbags 31 may be 1, 2, 4, 5 or more. In addition, the connection of the solenoid valves 55 and the inflating air pumps 51 to the corresponding first airbags 31 or second airbag 32 through a three-way value is only a specific embodiment, and those skilled in the art can adjust it as required so as to adapt to specific applications. For example, the first airbags 31 and the second airbag 32 can be each provided with an air inlet and an air outlet, the inflating air pump 51 is connected to the air inlet, and the solenoid valve 55 is connected to the air outlet; it is also possible that the first airbags 31 and the second airbag 32 are each provided with an air inlet-and-outlet port for air inflow and outflow, which is respectively connected to the inflating air pump 51 and the solenoid valve 55 through an electromagnetic three-way valve. The controller 6 controls the electromagnetic three-way valve to switch so that the air inlet-and-outlet port communicates with the inflating air pump 51 to inflate the first airbags 31 and the second airbag 32 or the air inlet-and-outlet port communicates with the atmosphere to deflate the first airbags 31 and the second airbag. In addition, inflating the outermost first airbag 31, the middle first airbag 32, the innermost first airbag 31 and the second airbag 32 in sequence is only a specific embodiment, and those skilled in the art can adjust it as required so as to adapt to specific applications such as inflating the three first airbags 31 to a set air pressure value at the same time, and then inflating the second airbag 32 to a set air pressure value, or inflating in other suitable ways. During the inflation process, the inflation speed may also be adjusted in real time according to a set air pressure curve. Of course, during the deflation process, the first airbags 31 and the second airbag 32 may also be deflated at the same time, or the second airbag 32 and the first airbags 31 from the inside to the outside may be deflated in sequence according to an air pressure curve. In addition, the air pump assembly including a plurality of first air pumps and each of the first air pumps being connected to the first airbags 31 and the second airbag 32 in a one-to-one correspondence is only a preferred embodiment, and those skilled in the art can adjust it as required so as to adapt to specific applications. For example, the air pump assembly only includes one first air pump, which is connected to the first airbags 31 and the second airbag 32 through a multi-way valve, and the first air pump communicates with any one of the first airbags 31 and the second airbag 32 respectively by controlling switching the multi-way valve.

With continued reference to FIG. 3, preferably, the air pump assembly includes four second air pumps, such as miniature vacuum pumps 52. The four miniature vacuum pumps 52 are respectively connected to the solenoid valves 55. The solenoid valves 55 and the inflating air pumps 51 are connected to the air inlet-and-outlet ports through the three-way valve 54, and the miniature vacuum pumps 52 communicate with the controller 6. After the pulse condition information is collected by the pulse diagnosis sensor 4, the controller 6 controls the solenoid valves 55 to open, and at the same time controls the miniature vacuum pumps 52 to open. The miniature vacuum pumps 52 are configured to pump out the air, thereby speeding up the deflation of the first airbags 31 and the second airbag 32, so that the first airbags 31 and the second airbag 32 are quickly restored to the initial state, which facilitates the wrist 8 to move out of the chamber 2 in time and further optimizes the user experience.

Preferably, the air pressures inside the first airbags 31 and the second airbag 32 are the same after being deflated by the miniature vacuum pumps 52. For example, after some the gas is discharged, a certain pressure is maintained inside the first airbags 31 and the second airbag 32, or after all the gas is discharged, a vacuum is formed in the first airbags 31 and the second airbag 32. The air pressures inside the first airbags 31 and the second airbag 32 are the same after being deflated, so that the first airbags 31 and the second airbag 32 can be inflated at the same inflation speed during inflation, which improves the stability of inflation and can prolong the service life of the inflating air pump 51.

In an alternative embodiment, the air pump assembly only includes four first air pumps, and the first air pumps are dual-purpose pumps capable of both inflating and deflating, which are connected to the air inlet-and-outlet ports. When the wrist 8 needs to be fixed, the controller 6 controls the dual-purpose pumps to inflate the first airbags 31 and/or the second airbag 32. After the pulse diagnosis sensor 4 collects the pulse condition information, the controller 6 controls the dual-purpose pumps to deflate the first airbags 31 and/or the second airbag 32. Through such an arrangement, the number of air pumps can be reduced, the structure can be simplified, and the cost can be reduced to a certain extent.

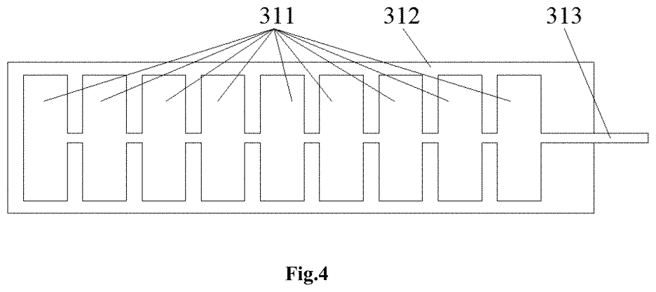

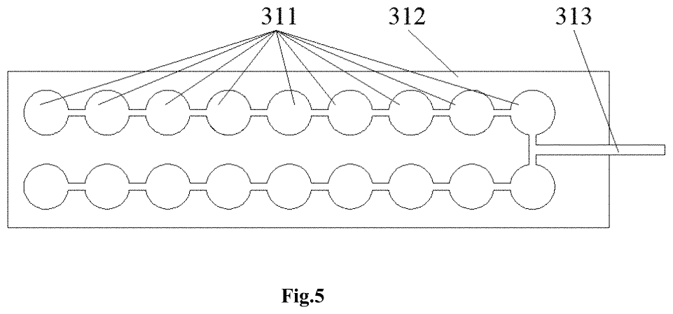

With reference to FIGS. 4 and 5 and with continued reference to FIGS. 1 and 2, preferably, each of the first airbags 31 is arranged around the chamber 2. As shown in FIGS. 1, 2, 4 and 5, the first airbag 31 is a belt-like airbag, and three belt-shaped airbags are arranged around the chamber 2. After the three first airbags 31 are inflated to the set pressure, the innermost first airbag 31 is wrapped around the wrist 8, which improves the fixing effect of the first airbag 31 on the wrist 8 and the comfort. It should be noted that a width of the first airbag 31 may be set by enlarging or reducing by a ratio of 20% according to the height of the shell 1, or may also be set according to other ratios, or may be set to equal to the height of the shell 1.

It can be understood by those skilled in the art that the arrangement of each first airbag 31 around the chamber 2 is only a preferred embodiment, and those skilled in the art can adjust it as required so as to adapt to specific applications. For example, with reference to the orientation shown in FIG. 1, a plurality of first airbags 31 and one second airbag 32 are stacked above and below the chamber 2 respectively from the outside to the inside, and the upper second airbag 32 is provided with a pulse diagnosis sensor 4 on a lower side thereof. The first airbags 13 may also be annular airbags connected end to end. The annular airbags are arranged around the chamber 2 or may be arranged in other suitable ways.

With continued reference to FIGS. 4 and 5, preferably, each first airbag 31 includes a plurality of communicating inflatable cavities 311. As shown in FIG. 4, the first airbag 31 includes a sealed portion 312. A plurality of square-shaped communicating inflatable cavities 311 are distributed from left to right in the sealed portion 312. A right end of the sealed portion 312 is provided with an air inlet-and-outlet port 313 communicating with the inflatable cavities 311.

By arranging the first airbag 31 to include a plurality of communicating inflatable cavities 311, when the first airbag 31 is inflated to a set pressure value, a contact area between the first airbag 31 and the adjacent object can be increased on the basis that the first airbag 31 reaches a set deformation amount, so that the forces on the wrist 8 and the pulse diagnosis sensor 4 are more uniform, and the comfort of fixing the wrist 8 and the accuracy of the pulse diagnosis sensor 4 are further improved.

It can be understood by those skilled in the art that the square shape of the inflatable cavity 311 is only an exemplary description, and those skilled in the art can adjust it as required so as to adapt to specific applications. For example, the shape of the inflatable cavity 311 may also be a triangle, a circle as shown in FIG. 5, or other suitable shapes. It can also be understood by those skilled in the art that the second airbag 32 may also be configured to include a plurality of communicating inflatable cavities.

With continued reference to FIGS. 1 and 2, preferably, the airbag assembly is equipped with a restoring unit to accelerate the speed of the first airbag 31 restoring to the initial state during the deflation process. Specifically, the restoring unit includes three elastic members arranged between the innermost first airbag 31 and the second airbag 32 as well as between the first airbag 31 and the first airbag 31, such as arc-shaped elastic strips 7. Two ends of the arc-shaped elastic strip 7 are overlapped to form a ring-like elastic structure. Two arc-shaped elastic strips 7 are respectively arranged between the three stacked first airbags 31, and the other arc-shaped elastic strip 7 is arranged between the innermost first airbag 31 and the second airbag 32.

In the process of inflating the first airbag 31, as the inflated gas increases in the first airbag 31, the deformation of the first airbag 31 increases, and the outermost first airbag 31 expands to generate a pressure toward the wrist 8 to the outermost arc-shaped elastic strip 7 so that the arc-shaped elastic strip 7 is deformed, and the two ends of the arc-shaped elastic strip 7 slide in opposite directions to gradually reduce the size of the ring-like elastic structure; the outermost arc-shaped elastic strip 7 simultaneously squeezes the middle first airbag 31, and the middle first airbag 31 expands and squeezes the middle arc-shaped elastic strips 7 to deform it so that the size of the formed ring-like structure is reduced. The middle arc-shaped elastic strip 7 squeezes the innermost first airbag 31, and the innermost first airbag 31 squeezes the innermost arc-shaped elastic strip 7 so that the innermost arc-shaped elastic strip 7 abuts against the surface of the wrist 8; at the same time, the innermost arc-shaped elastic strip 7 squeezes the second airbag 32, and the second airbag 32 squeezes the pulse diagnosis sensor 4, so that the pulse diagnosis sensor 4 abuts against the area corresponding to the radial artery vessel 81 on the wrist 8.

After the pulse diagnosis sensor 4 collects the pulse condition information, the controller 6 controls the solenoid valves 55 to open. In a case where no miniature vacuum pumps 52 are provided, the deformation of the three arc-shaped elastic strips 7 is restored and the first airbags 31 are squeezed so that the gas inside the first airbags 31 is discharged from the solenoid valves 55, which accelerates the deflation speed of the first airbags 31. In a case where the miniature vacuum pumps 52 are provided, as the deformation of the arc-shaped elastic strips 7 restores and squeezes the first airbags 31 to promote gas discharge, the miniature vacuum pumps 52 work to pump air, which further accelerates the deflation of the first airbags 31. In the process of inflating and deflating the first airbags 31, the two ends of the arc-shaped elastic strip 7 are overlapped, and the shape of the elastic strip remains the ring-like structure unchanged before, during and after deformation, so as to keep the forces on the surface of the wrist consistent, which avoids discomfort caused by uneven forces on the wrist and optimizes the user experience.

Through the arrangement of the restoring unit, the restoring of the first airbags 31 to the initial state can be accelerated, which facilitates the wrist to move out of the chamber in time, and further optimizes the user experience in use. It can be understood by those skilled in the art that the elastic member being an arc-shaped elastic strip 7 with two ends overlapped is only a specific embodiment, and those skilled in the art can adjust it as required so as to adapt to specific applications. For example, the elastic member may be an elastic ring with two ends fixedly connected, or a spring connected end to end, or other suitable elastic members. In addition, it is only a preferred embodiment that the restoring unit includes a plurality of elastic members arranged between the innermost first airbag 31 and the second airbag 32 as well as between the plurality of first airbags 31, and those skilled in the art can adjust it as required so as to adapt to specific applications. For example, the restoring unit can only include the elastic member arranged between the innermost first airbag 31 and the second airbag 32 or elastic members arranged in other ways, etc.

The control method for the pulse diagnosis instrument will be described in detail below with reference to a pulse diagnosis instrument according to an embodiment of the present disclosure.

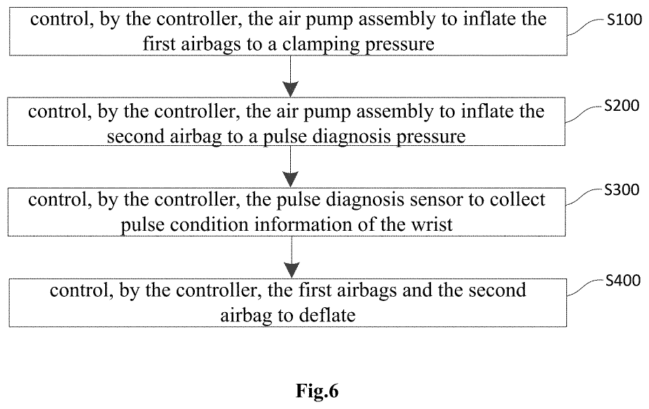

With reference to FIG. 6 and with continued reference to FIGS. 1 to 3, FIG. 6 is a schematic view showing steps of a control method for a pulse diagnosis instrument according to an embodiment of the present disclosure. As shown in FIG. 6, the control method for the pulse diagnosis instrument of the present disclosure mainly includes the following steps: S100: controlling, by the controller, the air pump assembly to inflate the first airbags to a clamping pressure; S200: controlling, by the controller, the air pump assembly to inflate the second airbag to a pulse diagnosis pressure; S300: controlling, by the controller, the pulse diagnosis sensor to collect pulse condition information of the wrist; and S400: controlling, by the controller, the first airbags and the second airbag to deflate.

Specifically, the controller 6 controls the air pump assembly to inflate the three first airbags 31 first so that the air pressures inside the three first airbags 31 reach a set clamping pressure, thus fixing the wrist 8; then the controller 6 controls the air pump assembly to inflate the second airbag 32 so that the air pressure inside the second airbag 32 reaches a set pulse diagnosis pressure, and then the controller 6 controls the pulse diagnosis sensor 4 to collect the pulse condition information of the wrist 8. Through this control method, the plurality of first airbags 31 can be filled with different amounts of gas to achieve different deformations and then clamp and fix the wrists 8 with different thicknesses at the set clamping pressure; then the second airbag 32 can be filled with a proper amount of gas to enable the second airbag 32 to reach a set pulse diagnosis pressure, so that the pulse diagnosis sensor 4 abuts against the area corresponding to the radial artery vessel 81 on the wrist 8 at an appropriate pressure, which greatly improves the accuracy of collecting the pulse condition information by the pulse diagnosis sensor 4.

Preferably, step S100 specifically includes: controlling, by the controller 6, the air pump assembly to sequentially inflate each of the first airbags 31 to the clamping pressure in an order from the outside to the inside. Specifically, the air pump assembly includes four first air pumps, such as inflating air pumps 51, and each of the first airbags 31 and the second airbag 32 is connected to one of the inflating air pumps 51 and a solenoid valve 55 through a three-way valve 54 respectively. The controller 6 is communicatively connected to the inflating air pumps 51, the air pressure sensors 53, the pulse diagnosis sensor 4 and the solenoid valves 55. The controller 6 controls the inflating air pump 51 connected to the outermost first airbag 31 to work and inflate. During the inflation process, the corresponding air pressure sensor 53 detects the air pressure value of the outermost first airbag 31 in real time. When the air pressure value reaches a set air pressure value, inflation of the outermost first airbag 31 is stopped. Then, the controller 6 controls the inflating air pump 51 connected to the middle first airbag 31 to work and inflate. During the inflation process, the corresponding air pressure sensor 53 detects the air pressure value of the middle first airbag 31 in real time. When the air pressure value reaches a set air pressure value, inflation of the middle first airbag 31 is stopped. Then, the controller 6 controls the inflating air pump 51 connected to the innermost first airbag 31 to work and inflate. During the inflation process, the corresponding air pressure sensor 53 detects the air pressure value of the innermost first airbag 31 in real time. When the air pressure value reaches a set air pressure value, inflation of the innermost first airbag 31 is stopped so that the innermost first airbag 31 abuts against the surface of the wrist 8 at a suitable pressure. Through such a control method, the first airbags 31 can be further slowly pressurized after abutting against the wrist 8, so that the comfort of the wrist 8 is improved on the basis of fixing the wrist 8. In addition, this control method enables the second airbag 32 and the pulse diagnosis sensor 4 to accurately abut against the area corresponding to the radial artery vessel 81 on the wrist 8, which improves the fitting accuracy of the sensor 4 and further improves the accuracy of collecting the pulse condition information by the pulse diagnosis sensor 4.

Preferably, step S400 specifically includes: controlling, by the controller 6, the first airbags 31 and the second airbag 32 to deflate at the same time. Through such a control method, rapid deflation of the first airbags 31 and the second airbag 32 can be realized, which facilitates the wrist 8 to move out of the chamber 2 in time and improves the operation efficiency.

In an alternative control method, step S400 specifically includes: controlling, by the controller 6, the second airbag 32 and the first airbags 31 to sequentially deflate in an order from the inside to the outside. In other words, the controller 6 first controls the second airbag 32 to deflate, then controls the innermost first airbag 31 to deflate, then controls the middle first airbag 31 to deflate, and finally controls the outermost first airbag 31 to deflate. Through such an arrangement, it is possible to first depressurize the area corresponding to the radial artery vessel 81 on the wrist 8, and then gradually depressurize the entire compressed area of the wrist 8 until the wrist 8 can move out of the chamber 2 freely, thereby avoiding a sudden change in blood pressure in the blood vessels inside the wrist 8, which would otherwise cause discomfort in the wrist 8, and optimizing the user experience.

Preferably, during the deflation process, the deflating and depressurizing can be performed according to a set depressurizing curve, so as to further improve the comfort and optimize the use experience. For example, the depressurizing curve is a stepped curve in which the pressure decreases in stages over time, and the deflating and depressurizing is performed in stages; the depressurizing curve may also be a curve in which the pressure decreases continuously over time, and the deflating continues until the air pressure decreases to the set value, etc.

Preferably, the air pressures of the first airbags 31 and the air pressure of the second airbag 32 are the same after deflation. For example, part of the gas in the first airbags 31 and the second airbag 32 can be released, or all the gas in the first airbags 31 and the second airbag 32 can be released to form a vacuum, so that the air pressures of the first airbags 31 and the air pressure of the second airbag 32 are the same. Through such an arrangement, the first airbags 31 and the second airbag 32 can be inflated at the same inflation speed during inflation, which improves the stability of inflation and prolongs the service life of the air pump assembly.

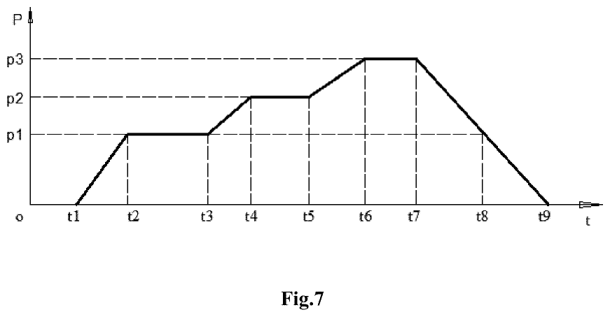

With reference to FIG. 7, FIG. 7 is a schematic view showing a change of pressure over time in the second airbag under the control of another control method for the pulse diagnosis instrument of the present disclosure.

In another specific embodiment, the control method for the pulse diagnosis instrument of the present disclosure includes the following steps: controlling, by the controller, the air pump assembly to inflate the first airbags to a first set pressure; controlling, by the controller, the air pump assembly to inflate the second airbag to a second set pressure; controlling, by the controller, the air pump assembly to inflate the first airbags so that an air pressure of the second airbag reaches a pulse diagnosis pressure; controlling, by the controller, the pulse diagnosis sensor to collect pulse condition information of the wrist; and controlling, by the controller, the first airbags and the second airbag to deflate.

Specifically, as shown in FIG. 7, the controller 6 first controls the inflating air pump 51 to inflate the first airbag 31, and stops inflating the first airbag 31 at time t1 so that the first airbag 31 reaches a first set pressure and a part of the first airbag 31 just touches the surface of the wrist 8; then the inflating air pump 51 inflates the second airbag 32, and stops inflating the second airbag 32 at time t2 so that the second airbag 32 reaches a second set pressure (such as p1); from time t2 to time t3, the inflation is not performed, so that the air pressure of the second airbag 32 is maintained at p1; from time t3, the first airbag 31 is inflated again and the inflation is stopped at time t4, and the second airbag 32 is squeezed by the first airbag 31 to make the air pressure of the second airbag 32 increase from p1 to p2; from time t4 to time t5, the inflation is not performed, so that the air pressure of the second airbag 32 is maintained at p2; from time t5 to time t6, the first airbag 31 is further inflated, and the second airbag 32 is squeezed by the first airbag 31 to make the air pressure of the second airbag 32 increase from p2 to p3 (i.e. the pulse diagnosis pressure); from time t6 to time t7, the inflation is not performed, so that the air pressure of the second airbag 32 is maintained at p3, and in this process, the pulse diagnosis sensor 4 is controlled to collect the pulse condition information of the wrist 8; from time t7 to time t8, the first airbag 31 is controlled to deflate to reduce the air pressure of the second airbag 32 from p3 to p1; and from time t8 to time t9, the second airbag 32 is controlled to deflate to reduce the air pressure of the second airbag 32 from p1 to the atmospheric pressure.

The control method of first inflating the first airbag 31, then inflating the second airbag 32 and finally inflating the first airbag 31 again can quickly make the pulse diagnosis sensor 4 on the second airbag 32 contact the wrist 8. During the inflation process of the second airbag 32, the pulse diagnosis sensor 4 can more accurately abut against the position corresponding to the radial artery vessel 81 on the wrist. Finally, the first airbag 31 is inflated again. As compared with the second airbag 32, the first airbag 31 has a larger volume. When the inflating air pump 51 is inflating at the minimum inflation speed, the volume of the first airbag 31 becomes larger at a slower speed, so that the air pressure of the second airbag 32 slowly increases, and further the pulse diagnosis sensor 4 abuts against the wrist at a smoothly changing pressure, which improves the accuracy of the pulse diagnosis sensor 4 abutting against the corresponding position on the wrist 8, and makes the pulse condition information collected by the pulse diagnosis sensor 4 more accurate. At the same time, the pressure on the wrist is prevented from increasing too fast and causing discomfort, and the user experience is improved. During the deflation process, the first airbag 31 is deflated first, and then the second airbag 32 is deflated, so that the pressure on the wrist 8 can change smoothly, thereby improving the comfort of the wrist 8 and optimizing the user experience.

It can be understood by those skilled in the art that it is only a specific embodiment that during the inflation process, after the second airbag 32 is inflated, the first airbag 31 is inflated in two stages to make the air pressure of the second airbag 32 reach the pulse diagnosis pressure, and those skilled in the art can adjust it as actually required so as to adapt to specific applications. For example, in the inflation process, after the second airbag 32 is inflated, the first airbag 31 can be inflated in one stage, three stages, four stages or more stages to make the air pressure of the second airbag 32 reach the pulse diagnose pressure. In addition, as shown in FIG. 7, during the inflation process, the air pressure of the second airbag 32 changes uniformly over time. Such a form of air pressure change is only a specific embodiment, and those skilled in the art can adjust it as actually required so as to adapt to specific applications. For example, the air pressure changing speed of the second airbag 32 over time may change in each stage. For example, it may be gradually reduced to form an air pressure-time changing curve, or it may be reduced in stages to form an air pressure-time changing fold line, or other suitable air pressure-time changing forms can be used, etc. Of course, the air pressure changing speed of the second airbag 32 during the deflation process may also change over time.

As can be seen from the above description, in the preferred technical solutions of the present disclosure, one or more first airbags and one second airbag stacked in sequence from the outside to the inside are arranged between the inner wall of the shell and the chamber, the first airbags and the second airbag are respectively equipped with an air pressure sensor, a side of the second airbag facing the chamber is provided with a pulse diagnosis sensor, and the controller can control the air pump assembly to separately inflate any one of the first airbags and the second airbag. The pulse diagnosis instrument also includes a plurality of first air pumps and a plurality of second air pumps. Each of the first air pumps is connected to the first airbags and the second airbag in a one-to-one correspondence, and each of the second air pumps is connected to the first airbags and the second airbag in a one-to-one correspondence. The first air pumps are configured to inflate the first airbags and the second airbag, and the second air pumps are configured to accelerate the deflation of the first airbags and the second airbag. The controller controls the first air pumps to inflate and deflate the first airbags and the second airbag, so that the airbag assembly can have a larger deformation range, thereby expanding the application range of the pulse diagnosis instrument. In addition, the cooperation of the first airbags and the second airbag enables the pulse diagnosis sensor to abut against the radial artery measurement area of the wrist with an appropriate force, which improves the clamping comfort to the wrist on the basis of ensuring the accuracy of collecting the pulse condition information. The first airbag is inflated first to the clamping pressure to fix the wrist, and then the second airbag is inflated to the pulse diagnosis pressure, so that the wrists with different thicknesses can be clamped and fixed, and at the same time, the accuracy of collecting the pulse condition information collection by the pulse diagnosis sensor can be improved.

Hitherto, the technical solutions of the present disclosure have been described in conjunction with the preferred embodiments shown in the accompanying drawings, but it is easily understood by those skilled in the art that the scope of protection of the present disclosure is obviously not limited to these specific embodiments. Without departing from the principles of the present disclosure, those skilled in the art can make equivalent changes or replacements to relevant technical features, and all the technical solutions after these changes or replacements will fall within the scope of protection of the present disclosure.

1. A pulse diagnosis instrument, comprising a shell, a chamber formed in the shell to accommodate a wrist, an airbag assembly provided between the chamber and an inner wall of the shell, a controller, and an air pump assembly connected to the airbag assembly,
wherein the airbag assembly comprises one or more first airbags and a second airbag stacked in sequence from the outside to the inside, the first airbags and the second airbag are each equipped with an air pressure sensor, and the second airbag is provided with a pulse diagnosis sensor on a side facing the chamber, and
wherein the controller can control the air pump assembly to separately inflate any one of the first airbags and the second airbag.

2. The pulse diagnosis instrument according to claim 1, wherein the air pump assembly comprises a plurality of first air pumps, and each of the plurality of first air pumps is connected to each of the first airbags and the second airbag in a one-to-one correspondence, and
wherein the first air pumps are configured to at least inflate the first airbags and the second airbag.

3. The pulse diagnosis instrument according to claim 2, wherein the air pump assembly further comprises a plurality of second air pumps, and each of the plurality of second air pumps is also connected to each of the first airbags and the second airbag in a one-to-one correspondence, and
wherein the second air pumps are configured to accelerate the deflation of the first airbags and the second airbag.

4. The pulse diagnosis instrument according to claim 3, wherein the first airbags and the second airbag are each provided with an air inlet-and-outlet port, and each of the air inlet-and-outlet ports is connected to a corresponding first air pump and second air pump respectively through a three-way valve; or
the first airbags and the second airbag are each provided with an air inlet and an air outlet, the air inlet is connected to a corresponding first air pump, and the air outlet is connected to a corresponding second air pump.

5. The pulse diagnosis instrument according to claim 2, wherein the first air pumps are dual-purpose pumps capable of both inflating and deflating.

6. The pulse diagnosis instrument according to claim 4, wherein each of the first airbags is arranged around the chamber.

7. The pulse diagnosis instrument according to claim 6, wherein each of the first airbags comprises a plurality of communicating inflatable cavities.

8. The pulse diagnosis instrument according to claim 2, wherein the airbag assembly is equipped with a restoring unit to accelerate the speed of the first airbag restoring to an initial state during the deflation process.

9. The pulse diagnosis instrument according to claim 8, wherein the restoring unit comprises an elastic member arranged between the innermost first airbag and the second airbag; or
the restoring unit comprises a plurality of elastic members arranged between the innermost first airbag and the second airbag as well as between the plurality of first airbags.

10. The pulse diagnosis instrument according to claim 9, wherein the elastic member is an arc-shaped elastic strip with both ends overlapped.

11. A control method for a pulse diagnosis instrument, wherein the pulse diagnosis instrument comprises a shell, a chamber formed in the shell to accommodate a wrist, an airbag assembly provided between the chamber and an inner wall of the shell, a controller, and an air pump assembly connected to the airbag assembly;
the airbag assembly comprises one or more first airbags and a second airbag stacked in sequence from the outside to the inside, the first airbags and the second airbag are each equipped with an air pressure sensor, and the second airbag is provided with a pulse diagnosis sensor on a side facing the chamber;
wherein the control method comprises the following steps:
controlling, by the controller, the air pump assembly to inflate the first airbags to a clamping pressure;
controlling, by the controller, the air pump assembly to inflate the second airbag to a pulse diagnosis pressure;
controlling, by the controller, the pulse diagnosis sensor to collect pulse condition information of the wrist; and
controlling, by the controller, the first airbags and the second airbag to deflate.

12. The control method according to claim 11, wherein the step of “controlling by the controller the air pump assembly to inflate the first airbags to the clamping pressure” specifically comprises:
controlling, by the controller, the air pump assembly to sequentially inflate each of the first airbags to the clamping pressure in an order from the outside to the inside.

13. The control method according to claim 11, wherein the step of “controlling by the controller the first airbags and the second airbag to deflate” specifically comprises:
controlling, by the controller, the first airbags and the second airbag to deflate at the same time.

14. The control method according to claim 11, wherein the step of “controlling by the controller the first airbags and the second airbag to deflate” specifically comprises:
controlling, by the controller, the second airbag and the first airbags to sequentially deflate in an order from the inside to the outside.

15. The control method according to claim 13, wherein the air pressures of the first airbags and the air pressure of the second airbag are the same after deflation.

16. A control method for a pulse diagnosis instrument, wherein the pulse diagnosis instrument comprises a shell, a chamber formed in the shell to accommodate a wrist, an airbag assembly provided between the chamber and an inner wall of the shell, a controller, and an air pump assembly connected to the airbag assembly;
the airbag assembly comprises one or more first airbags and a second airbag stacked in sequence from the outside to the inside, the first airbags and the second airbag are each equipped with an air pressure sensor, and the second airbag is provided with a pulse diagnosis sensor on a side facing the chamber;
wherein the control method comprises the following steps:
controlling, by the controller, the air pump assembly to inflate the first airbags to a first set pressure;
controlling, by the controller, the air pump assembly to inflate the second airbag to a second set pressure;
controlling, by the controller, the air pump assembly to inflate the first airbags so that an air pressure of the second airbag reaches a pulse diagnosis pressure;
controlling, by the controller, the pulse diagnosis sensor to collect pulse condition information of the wrist; and
controlling, by the controller, the first airbags and the second airbag to deflate.
